(19) United States
(12) Patent Application Publication
(10) Pub. No.: US 2023/0374728 A1
(43) Pub. Date: Nov. 23, 2023

(54) COLLAGEN BIOMATERIAL

(71) Applicant: Hide Biotech Ltd, London (GB)

(72) Inventor: Yudi Ding, Cambridge, Cambridgeshire (GB)

(21) Appl. No.: 18/248,392

(22) PCT Filed: Oct. 12, 2021
(86) PCT No.: PCT/GB2021/052633

§ 371 Date: Apr. 10, 2023

(30) Foreign Application Priority Data
Oct. 13, 2020 (GB) 2016195.6

(51) Int. Cl. D06N 3/00; C12N 9/10

(52) U.S. Cl. CPC D06N 3/0018; C12N 9/1044; D06N 2211/28; C12Y 203/02013

(57) ABSTRACT

The present invention describes a biomaterial made from a collagen composition comprising (i) partially hydrolyzed collagen and (ii) collagen and/or fully hydrolyzed collagen. The biomaterial is useful in producing a processed biomaterial which may be a leather-like material.

### CROSS-REFERENCE TO RELATED APPLICATIONS

This application is a 371 National Phase Entry of International Patent Application No. PCT/GB2021/052633 filed on Oct. 12, 2021, which claims the benefit of British Provisional Patent Application No. 2016195.6 filed on Oct. 13, 2020, the contents of which are incorporated herein by reference in their entirety.

### FIELD OF THE INVENTION

The invention relates to a method of producing a biomaterial using a collagen composition, and a biomaterial comprising a collagen composition. The collagen composition may be extracted from a marine product.

### BACKGROUND OF THE INVENTION

Leather is a widely used material and there is a huge global demand for leather products. For example, leather is used in furniture upholstery, clothing, shoes, luggage, handbags, and accessories.

Natural leather is produced by the tanning of animal rawhide and skin, often cattle hide. Animal hide (and thus the leather made from animal hide) is formed mainly of collagen, a fibrous protein. Collagen is a generic term for a family of at least 28 distinct collagen types, which are all characterized by a repeating triplet of amino acids, -(Gly-X—Y),,—, so that approximately one third of the amino acid residues in collagen are glycine. X is often proline and Y is often hydroxyproline. Thus, the structure of collagen may consist of entwined triple units of peptide chains of differing lengths. Triple helices may be bound together in bundles called fibrils, and fibril bundles can come together to create fibers. The collagen fibers typically join with each other throughout a layer of skin. Crosslinking or linking may provide strength to the material.

The properties of natural leather are affected by the type of animal hide that is used. In particular, different animals produce different amino acid compositions of the collagen, which may result in different properties. Variations in collagen structure also occur throughout the thickness of the hide. The top grain side of hide is generally composed of a fine network of collagen fibrils while deeper sections (also known as the corium) are composed of larger fiber bundles. The top grain surface of leather is smoother and softer than the corium. Therefore, in order to produce natural leather with smooth grain on both sides, it is necessary to combine two pieces of grain, corium sides together, and either sew them together or laminate them with adhesives. There is a demand for a leather material in which the collagen structure can be controlled so as to produce a smooth surface on both sides to avoid this combination step.

The postprocessing steps used in leather manufacture are also limited by the natural variation in collagen structure between different animal hides. Although the final properties of leather can be controlled to some extent through the incorporation of stabilizing and lubricating molecules into the hide during the tanning stage, the selection of these molecules is limited by the need to penetrate the dense structure of the hide. There is a need for a method of producing leather materials in which the original collagen structure of the hide does not limit the postprocessing steps that can be used.

Alternative methods of making leather-like materials known in the art include culturing collagen to produce sheets which can then be crosslinked to produce a leather-like material. However, such methods are typically not very efficient and are difficult to implement in large scale production. In addition, leather-like materials made purely from collagen are typically not very strong.

Accordingly, there is a need to develop new biomaterials that may be processed to make improved leather-like biomaterials, and methods of creating leather-like biomaterials.

### SUMMARY OF THE INVENTION

The present inventors have found that collagen compositions comprising (i) at least 30% by weight of partially hydrolyzed collagen and (ii) collagen and/or fully hydrolyzed collagen can be used to make an improved biomaterial. This biomaterial may provide improved leather-like materials compared to those known in the art. Previously known biomaterials made from collagen compositions use only collagen, and generally require time- and resource-heavy processing steps to make, for example high volumes of acidic solvents. The manufacture of such biomaterials made only from collagen also typically requires the handling of very thick and viscous collagen gels. This has the disadvantage that bubbles may form in the gel during processing which are difficult to remove and can lead to defects in the material. Previously known biomaterials also typically have low tensile strength. These factors mean that the biomaterials may be unsuitable for further processing into leather-like biomaterials with a high strength and smooth appearance. The present inventors, however, have found that biomaterials made from collagen compositions comprising (i) at least 30% by weight of partially hydrolyzed collagen and (ii) collagen and/or fully hydrolyzed collagen are stronger than previously known biomaterials made from collagen alone. Furthermore, the biomaterial can be produced more efficiently and using fewer resources, and bubbles can be more easily removed during the manufacturing process.

The present invention therefore provides a biomaterial comprising a dehydrated collagen gel, wherein the collagen gel comprises a collagen composition comprising (i) partially hydrolyzed collagen and (ii) collagen and/or fully hydrolyzed collagen, wherein the collagen composition comprises at least 30% by weight of partially hydrolyzed collagen.

Also provided is a method for producing the biomaterial of the invention, the method comprising: (a) forming the collagen composition into a collagen gel; and (b) dehydrating the collagen gel to form the biomaterial.

Also provided is a biomaterial comprising a dehydrated collagen gel, wherein the collagen gel comprises a collagen composition extracted from a marine product, and wherein the collagen composition comprises collagen and partially hydrolyzed collagen, and optionally fully hydrolyzed collagen.

Also provided is a leather-like processed biomaterial comprising the biomaterial described herein.

### DETAILED DESCRIPTION OF THE INVENTION

### Collagen Composition

The biomaterial of the present invention comprises a dehydrated collagen gel, which is formed from a collagen composition. As used herein, a collagen composition is any composition which comprises collagen or any collagen derivative (such as partially hydrolyzed collagen or fully hydrolyzed collagen). According to the present invention, the collagen composition comprises (i) partially hydrolyzed collagen, and (ii) collagen and/or fully hydrolyzed collagen.

As used herein, collagen refers to collagen in a triple helix structure. The collagen may be acid-soluble collagen. Partially hydrolyzed collagen refers to collagen which does not contain a triple helix structure but still contains amino acid chains. Typically, partially hydrolyzed collagen refers to single chain collagen. The partially hydrolyzed collagen may comprise gelatin. Typically, as used herein partially hydrolyzed collagen is gelatin. Fully hydrolyzed collagen refers to collagen peptides and/or amino acids. The fully hydrolyzed collagen may comprise collagen hydrolysate. Typically, as used herein fully hydrolyzed collagen is collagen hydrolysate.

The collagen, partially hydrolyzed collagen and fully hydrolyzed collagen may each independently originate from any animal source or product. Alternatively, the collagen, partially hydrolyzed collagen and fully hydrolyzed collagen may be prepared by in vitro synthetic procedures. As used herein, collagen or a collagen derivative (such as partially hydrolyzed collagen or fully hydrolyzed collagen) which originates from a particular animal source or product means a collagen-containing component which is extracted as part of a collagen composition from the animal source or product, optionally having been further processed (for example, hydrolyzed or purified) to produce the collagen or collagen derivative. For example, partially hydrolyzed collagen which originates from an animal source or product is originally extracted as part of a collagen composition from the animal source or product and is then obtained through hydrolysis of that collagen composition.

For example, the collagen, partially hydrolyzed collagen, and fully hydrolyzed collagen may each independently originate from a marine product, a bovine product or a porcine product, preferably a marine product or a porcine product. As used herein, collagen (or partially hydrolyzed or fully hydrolyzed collagen) which originates from a marine, bovine or porcine source or product may also be referred to as marine, bovine or porcine collagen (or partially hydrolyzed or fully hydrolyzed collagen), respectively. Typically, at least one of the partially hydrolyzed collagen, collagen and fully hydrolyzed collagen composition originates from a marine product. For example, in one embodiment the partially hydrolyzed collagen is partially hydrolyzed marine collagen. In one embodiment, the fully hydrolyzed collagen is fully hydrolyzed marine collagen. In one embodiment, the collagen is not bovine collagen. In one embodiment, none of the collagen, partially hydrolyzed collagen nor fully hydrolyzed collagen originate from a bovine product. The collagen, partially hydrolyzed collagen and/or fully hydrolyzed collagen in a particular collagen composition may all originate from the same type of animal source, or they may originate from different types of animal source. For example, in one embodiment the collagen composition comprises partially hydrolyzed marine collagen and porcine collagen.

The animal source or product may be any part of an animal which contains collagen. For example, the marine product may be any part of a marine animal which contains collagen. As used herein, a marine animal may be any animal that exists primarily or exclusively in a water-based environment and may include animals that are found in freshwater environments as well as in oceans. The marine animal may be a fish such as a bass, bream, brill, bull huss, catfish, coalfish, cod, dab, dogfish, eel, flounder, garfish, haddock, halibut, mackerel, plaice, pollock, ray, salmon, sardine, skate, smooth-hound, sole, tilapia, or tuna. The marine animal may be an invertebrate such as an anemone, clam, coral, hydrozoan, jellyfish, mussel, oyster, scallop, sea cucumber, sea slug, sea snail, sea urchin, sponge, starfish, or worm. The marine animal may be an arthropod such as an arachnid, crustacean, insect, or myriapod.

The marine product may be a freshwater fish product, a saltwater fish product, an invertebrate product or an arthropod product. In one embodiment, the marine product is a fish product, including freshwater fish as well as saltwater fish. A fish product may be any part of a fish which contains collagen. Typically, the fish product comprises one or more of fish skin, fish scale, a fish swim bladder, or fish joints and/or tendons, for example it may comprise one or more of fish skin, fish scale and/or fish swim bladder. All of these fish products contain collagen, although collagen content is particularly high in fish swim bladder which is used in one preferred embodiment. The fish product comprises fish skin in another preferred embodiment. Fish products are more sustainable than similar collagen-containing products from other animals e.g., bovine products, because the production of fish products requires less water and has a lower carbon footprint. In particular, fish skin is a conveniently accessible waste product and therefore use of fish skin has environmental benefits.

The bovine product may be any part of a bovine animal which contains collagen. In one embodiment, the bovine product is bovine tendon. The bovine animal may be a cow, a bison, a buffalo, or an antelope. Typically, the bovine product is a cow product. The porcine product may be any part of a porcine animal which contains collagen. In one embodiment, the porcine product is porcine skin. The porcine animal may be a pig, a hog, or a boar.

According to the present invention, the collagen composition comprises at least 30% by weight of partially hydrolyzed collagen. The presence of partially hydrolyzed collagen (such as gelatin) in the collagen composition improves the strength of the biomaterial and any processed biomaterial produced from the biomaterial. As used herein, reference to a % by weight of collagen or a collagen derivative means the weight of the collagen or collagen derivative expressed as a percentage of the weight of all the collagen or a collagen derivative component in the collagen composition.

The collagen composition may comprise at least 40%, at least 50%, at least 60%, at least 70%, at least 75%, at least 80%, at least 85%, at least 90%, or at least 95% by weight of partially hydrolyzed collagen. Preferably, the collagen composition comprises at least 70%, at least 75%, at least 80%, at least 85%, or at least 90% by weight of partially hydrolyzed collagen, more preferably at least 70% or at least 80% by weight of partially hydrolyzed collagen. Typically, the collagen composition comprises less than 99% by weight of partially hydrolyzed collagen, for example no more than 95% by weight of partially hydrolyzed collagen. The collagen composition may comprise from 30% to 95%, from 50% to 95%, from 60% to 95%, from 70% to 95%, from 75% to 95%, or from 80% to 95% by weight of partially hydrolyzed collagen. Alternatively, the collagen composition may comprise from 30% to 90%, from 50% to 90%, from 60% to 90%, from 70% to 90%, from 75% to 90%, or from 80% to 90% by weight of partially hydrolyzed collagen. Alternatively, the collagen composition may comprise from 30% to 85%, from 50% to 85%, from 60% to 85%, from 70% to 85%, from 75% to 85%, or from 80% to 85% by weight of partially hydrolyzed collagen.

The collagen composition comprises collagen and/or fully hydrolyzed collagen. The collagen composition may comprise at least 1% by weight of one or a mixture of collagen and/or fully hydrolyzed collagen. Typically, the collagen composition comprises at least 2%, at least 3%, at least 4%, or at least 5% by weight of one or a mixture of collagen and/or fully hydrolyzed collagen, preferably at least 5%. The collagen composition may comprise at least 10%, at least 15%, at least 20%, at least 30%, at least 40%, or at least 50% by weight of a mixture of collagen and/or fully hydrolyzed collagen. The collagen composition may comprise no more than 70%, no more than 60%, no more than 50%, no more than 40%, no more than 30%, no more than 25%, no more than 20%, no more than 15%, or no more than 10% by weight of one or a mixture of collagen and/or fully hydrolyzed collagen. Typically, the collagen composition comprises from 5% to 70%, from 5% to 50%, from 5% to 40%, from 5% to 30%, from 5% to 25%, or from 5% to 20% by weight of one or a mixture of collagen and/or fully hydrolyzed collagen. Alternatively, the collagen composition may comprise from 10% to 70%, from 10% to 50%, from 10% to 40%, from 10% to 30%, from 10% to 25%, or from 10% to 20% by weight of one or a mixture of collagen and/or fully hydrolyzed collagen. Alternatively, the collagen composition may comprise from 15% to 70%, from 15% to 50%, from 15% to 40%, from 15% to 30%, from 15% to 25%, or from 15% to 20% by weight of one or a mixture of collagen and/or fully hydrolyzed collagen.

The collagen composition may comprise at least 1% by weight of collagen and/or at least 1% by weight of fully hydrolyzed collagen. The collagen composition may comprise at least 2%, at least 3%, at least 4% or at least 5% by weight of one or both of collagen and fully hydrolyzed collagen. Preferably, the collagen composition comprises at least 5% by weight of collagen and/or at least 5% by weight of fully hydrolyzed collagen.

Where the collagen composition contains collagen, the collagen composition may comprise at least 1%, at least 5%, at least 10%, at least 15%, at least 20%, or at least 25% by weight of collagen, preferably at least 5% or at least 10% by weight of collagen. Typically, where the collagen composition contains collagen, the collagen composition comprises no more than 70% by weight of collagen, for example no more than 60%, no more than 50%, no more than 40%, no more than 30%, no more than 25%, no more than 20%, no more than 15%, or no more than 10% by weight of collagen. Preferably, the collagen composition comprises no more than 50% by weight of collagen. The collagen composition may comprise no more than 30% by weight of collagen. Typically, the collagen composition comprises from 5% to 70%, from 5% to 50%, from 5% to 40%, from 5% to 30%, from 5% to 25%, or from 5% to 20% by weight of collagen. Preferably, the collagen composition comprises from 5% to 50% or from 5% to 30% by weight of collagen. Alternatively, the collagen composition may comprise from 10% to 70%, from 10% to 50%, from 10% to 40%, from 10% to 30%, from 10% to 25%, or from 10% to 20% by weight of collagen. Alternatively, the collagen composition may comprise from 15% to 70%, from 15% to 50%, from 15% to 40%, from 15% to 30%, from 15% to 25%, or from 15% to 20% by weight of collagen. In one embodiment, the collagen mix does not contain collagen. Limiting the amount of collagen in the collagen composition enables the composition to contain more partially hydrolyzed collagen, which has been found by the present inventor to improve the tensile strength of the biomaterial and to increase the efficiency of the manufacturing method (in particular, by reducing or removing the neutralization that is required, and improving the handling of the product during manufacture, in particular by reducing the viscosity of the product and thereby reducing bubble formation). The use of less collagen also reduces the cost of the resulting biomaterial.

Where the collagen mix contains fully hydrolyzed collagen, the collagen mix may comprise at least 1%, at least 5%, at least 10%, at least 15%, at least 20%, or at least 25% by weight of fully hydrolyzed collagen. The presence of fully hydrolyzed collagen (for example collagen hydrolysate) in the collagen composition may improve the hardness, elasticity, ductility and strength of the biomaterial and any processed biomaterial produced from the biomaterial. However, it is important to control the amount of fully hydrolyzed collagen in the collagen composition because high amounts of fully hydrolyzed collagen weaken the collagen gel and the resulting biomaterial and prevent the collagen gel from holding together well. Where the collagen composition contains fully hydrolyzed collagen, typically the collagen composition comprises no more than 50%, no more than 40%, no more than 30%, no more than 25%, no more than 20%, no more than 15% or no more than 10% by weight of fully hydrolyzed collagen. Preferably, the collagen composition comprises no more than 30% or no more than 20% by weight of fully hydrolyzed collagen. Typically, the collagen composition comprises from 5% to 50%, from 5% to 40%, from 5% to 30%, from 5% to 25%, or from 5% to 20% by weight of fully hydrolyzed collagen. Preferably, the collagen composition comprises from 5% to 30% or from 5% to 20% by weight of fully hydrolyzed collagen. Alternatively, the collagen composition may comprise from 10% to 50%, from 10% to 40%, from 10% to 30%, from 10% to 25%, or from 10% to 20% by weight of fully hydrolyzed collagen. Alternatively, the collagen composition may comprise from 15% to 50%, from 15% to 40%, from 15% to 30%, from 15% to 25%, or from 15% to 20% by weight of fully hydrolyzed collagen. In one embodiment, the collagen mix does not contain fully hydrolyzed collagen.

In one embodiment, the collagen composition comprises (i) from 30% to 95% by weight of partially hydrolyzed collagen, and (ii) from 5% to 70% by weight of one or a mixture of collagen and/or fully hydrolyzed collagen. In one embodiment, the collagen composition comprises (i) from 50% to 95% by weight of partially hydrolyzed collagen, and (ii) from 5% to 50% by weight of one or a mixture of collagen and/or fully hydrolyzed collagen. In one embodiment, the collagen composition comprises (i) from 60% to 95% by weight of partially hydrolyzed collagen, and (ii) from 5% to 40% by weight of one or a mixture of collagen and/or fully hydrolyzed collagen. In one preferred embodiment, the collagen composition comprises (i) from 70% to 95% by weight of partially hydrolyzed collagen, and (ii) from 5% to 30% by weight of one or a mixture of collagen and/or fully hydrolyzed collagen. In one preferred embodiment, the collagen composition comprises (i) from 80% to 95% by weight of partially hydrolyzed collagen, and (ii) from 5% to 20% by weight of one or a mixture of collagen and/or fully hydrolyzed collagen. In one preferred embodiment, the collagen composition comprises (i) from 70% to 90% by weight of partially hydrolyzed collagen, and (ii) from 10% to 30% by weight of one or a mixture of collagen and/or fully hydrolyzed collagen.

### Extracted Collagen Composition

In one embodiment, the collagen gel may be formed from a collagen composition which is extracted from an animal product, in particular a marine product. As used herein, a collagen composition extracted from an animal (for example, marine) product may be described as an extracted collagen composition. As used herein, a collagen composition extracted from a marine product may be described as a marine collagen composition. A marine product as referred to herein is described above.

Advantageously, a collagen composition extracted from a marine product can be used efficiently to make a biomaterial which is well suited to further processing steps to create a leather-like biomaterial. Previously known methods for producing biomaterials from collagen do not use marine products as the collagen source and frequently require long and complicated extraction steps to provide collagen in a form suitable for further processing. Cultured collagen has also been used, but this is not efficient and the process is not easily scalable. Cultured collagen also has the disadvantage that it does not provide an endogenous mixture of natural collagen proteins.

The collagen in the extracted collagen composition may be extracted using an acid (i.e., acid-soluble collagen) or pepsin (i.e., pepsin-soluble collagen). Partially and/or fully hydrolyzed collagen may be added to the extracted collagen composition to give an extracted collagen composition with higher amounts of partially and/or fully hydrolyzed collagen. For example, partially hydrolyzed collagen and/or fully hydrolyzed collagen may be added to the extracted collagen composition to provide a collagen composition which contains (i) at least 30% by weight of partially hydrolyzed collagen and (ii) collagen and/or fully hydrolyzed collagen. Preferred amounts of collagen and/or fully hydrolyzed collagen are as described above. In one embodiment, the collagen composition as defined herein comprises an extracted collagen composition, for example a collagen composition extracted from a marine product.

The presence of partially and/or fully hydrolyzed collagen in the extracted collagen composition is useful in producing biomaterial. In particular, the partially and/or fully hydrolyzed collagen may increase the efficiency of crosslinking and gel formation. Furthermore, the partially and/or fully hydrolyzed collagen may improve the properties of the biomaterial and any processed biomaterial produced from the biomaterial. In particular, the presence of partially hydrolyzed collagen may result in a softer and more elastic biomaterial and/or processed biomaterial compared to biomaterials which do not contain partially hydrolyzed collagen.

The extracted collagen composition typically comprises collagen and optionally partially and/or fully hydrolyzed collagen. Typically, the extracted collagen composition comprises at least one of acid-soluble collagen, partially hydrolyzed collagen and fully hydrolyzed collagen. As used herein, acid-soluble collagen is collagen that is extractable using acid. The extracted collagen composition may contain at least 20%, at least 30%, or at least 40% by weight of acid-soluble collagen. For example, the extracted collagen composition may contain from 20% to 50% by weight of acid-soluble collagen. The extracted collagen composition may contain at least 1%, at least 5%, at least 10%, at least 15%, at least 20%, or at least 30% by weight of partially hydrolyzed collagen. For example, the extracted collagen composition may contain from 1% to 40% by weight of partially hydrolyzed collagen. The extracted collagen composition may contain at least 1%, at least 5%, at least 10%, at least 15%, at least 20%, or at least 30% by weight of fully hydrolyzed collagen. For example, the extracted collagen composition may contain from 1% to 40% by weight of fully hydrolyzed collagen. In one embodiment, the extracted collagen composition contains at least 20% by weight of acid-soluble collagen, and/or at least 1% by weight of partially hydrolyzed collagen, and/or at least 1% by weight of fully hydrolyzed collagen. In one embodiment, the extracted collagen composition contains at least 1% by weight of partially hydrolyzed collagen, and/or at least 1% by weight of fully hydrolyzed collagen. The amounts provided above for acid-soluble collagen may also be applied to collagen.

The extracted collagen composition may be an endogenous composition i.e., it contains collagen, collagen derivatives (such as partially and/or fully hydrolyzed collagen) and other components (such as naturally occurring impurities) as they are found naturally in the marine product. For example, the collagen will typically have the telopeptide regions intact. This may make the products formed from the extracted collagen composition more desirable to certain consumer groups. Advantageously, a collagen composition extracted from a marine product can be used in its endogenous form without requiring complicated processing. Furthermore, the endogenous composition may contain collagen derivatives which can improve the efficiency of biomaterial manufacture, and advantageously affect the properties of any processed biomaterial produced from the biomaterial.

### Methods of Manufacture

The invention also relates to a method of manufacturing the biomaterial described herein. Typically, the method for producing the biomaterial comprises: (a) forming a collagen composition into a collagen gel; and (b) dehydrating the collagen gel to form the biomaterial.

The collagen composition may be any collagen composition as defined herein. In one embodiment, the invention provides a method for producing a biomaterial, the method comprising: (a) forming a collagen composition into a collagen gel; and (b) dehydrating the collagen gel to form the biomaterial; wherein the collagen composition comprises (i) partially hydrolyzed collagen, and (ii) collagen and/or fully hydrolyzed collagen, wherein the collagen composition comprises at least 30% by weight of partially hydrolyzed collagen.

In one embodiment, the forming step (a) comprises contacting the collagen composition with one or more crosslinking agents to form a crosslinkable collagen mixture and crosslinking the crosslinkable collagen mixture to form the collagen gel.

The forming step may comprise adding a fatliquoring component and/or a dye or pigment to the collagen composition or crosslinkable collagen mixture. In one embodiment, the invention provides a method for producing the biomaterial of the invention, the method comprising: (a) forming a collagen composition into a collagen gel; and (b) dehydrating the collagen gel to form the biomaterial; wherein the forming step comprises adding a fatliquoring component and/or a dye or pigment to the collagen composition.

In one embodiment, the invention provides a method for producing the biomaterial of the invention, the method comprising: (a) forming a collagen composition into a collagen gel; and (b) dehydrating the collagen gel to form the biomaterial; wherein the forming step comprises contacting the collagen composition with one or more crosslinking agents to form a crosslinkable collagen mixture, and crosslinking the crosslinkable collagen mixture to form the collagen gel, and wherein the forming step further comprises adding a fatliquoring component and/or a dye or pigment to the collagen composition or crosslinkable collagen mixture.

Also disclosed herein is a method for producing a biomaterial, the method comprising: (a) extracting a collagen composition from an animal product, preferably a marine product; and (b) forming the collagen composition into a collagen gel and dehydrating the collagen gel to form the biomaterial; wherein the collagen composition comprises collagen and optionally partially and/or fully hydrolyzed collagen.

The extraction step (a) may also comprise adding partially hydrolyzed and/or fully hydrolyzed collagen to the extracted collagen composition, to provide a collagen composition which comprises (i) partially hydrolyzed collagen, and (ii) collagen and/or fully hydrolyzed collagen, wherein the collagen composition comprises at least 30% by weight of partially hydrolyzed collagen. In one embodiment, the collagen composition comprises partially hydrolyzed collagen, collagen, and optionally fully hydrolyzed collagen, wherein the collagen composition comprises at least 30% by weight of partially hydrolyzed collagen.

Any of the methods described herein may further comprise processing the biomaterial to form a processed biomaterial. The processed biomaterial may be a leather-like biomaterial. The processing step(s) may include one or more of drying, dyeing, fatliquoring, finishing, and coating the biomaterial.

In one embodiment, the extraction step (a) comprises washing the animal product, for example a marine product, with an alkaline solution, optionally further washing the marine product with a degreasing agent, contacting the marine product with an acidic solution of pH 4 to 5, and obtaining an extracted collagen composition from the acidic solution; the forming step (b) comprises contacting the extracted collagen composition with one or more crosslinking agents to form a crosslinkable collagen mixture, crosslinking the crosslinkable collagen mixture to form the collagen gel, and dehydrating the collagen gel; and optionally the method further comprises a processing step (c) after the forming step (b) to form a processed biomaterial, wherein the processing step comprises fatliquoring the biomaterial, dying the fatliquored biomaterial, drying the dyed and fatliquored biomaterial, and mechanically working the dried biomaterial.

Also described herein is a biomaterial, wherein the biomaterial is obtainable by the methods as described herein. The biomaterial may be a leather-like biomaterial.

(a) Extraction

The section below describes the extraction of a collagen composition from an animal product. References to an animal product herein may be taken as references to a specific animal product (e.g., a marine product) where the collagen is extracted from that specific animal product.

The collagen may be extracted from the animal product, for example a marine product, by contacting the animal product with an acidic solution. The acidic solution may be any solution with a pH of less than 7. Typically, the acidic solution may have a pH of 3 to 6, preferably from 4 to 6. In a preferred embodiment, the acidic solution has a pH of 4 to 5. The acidic solution may comprise any weak acid or diluted strong acid with an appropriate pH. Typically, the acidic solution is an aqueous solution of acetic acid, formic acid, or hydrochloric acid. In one embodiment, the acidic solution is an aqueous solution of acetic acid.

The animal product may be contacted with the acidic solution for at least 6 hours, or at least 12 hours, or at least 24 hours. Typically, the animal product is contacted with the acidic solution for about 12 hours. The animal product may be contacted with the acidic solution at a temperature of less than 30° C., or less than 20° C., or less than 10° C. Typically, the animal product is contacted with the acidic solution at a temperature of around 4° C. After contacting the animal product with the acidic solution, the extracted collagen composition may be separated from the solution. The extracted collagen composition may be freeze dried (lyophilized).

The extraction step may further comprise washing the animal product with an alkaline solution before the animal product is contacted with the acidic solution. The animal product may be washed with the alkaline solution more than once. For example, the animal product may be washed with the alkaline solution twice. The alkaline solution may be useful in removing noncollagen proteins from the animal product and in breaking down the animal product. The alkaline solution may be any solution with a pH of more than 7. Typically, the alkaline solution is an aqueous solution of sodium hydroxide, potassium hydroxide, sodium carbonate or magnesium carbonate. In one embodiment, the alkaline solution is a solution of sodium hydroxide.

The extraction step may further comprise washing the animal product with a degreasing agent before the animal product is contacted with the acidic solution. The degreasing agent may be useful in removing fat from the product. Typically, the degreasing agent is an alcohol solution, an organic solvent (such as chloroform, petroleum ether, or n-hexane) or supercritical CO₂. In a preferred embodiment, the degreasing agent is an alcohol solution. The alcohol solution may contain less than 70% v/v, less than 50% v/v, or less than 30% v/v alcohol in water. Typically, the alcohol solution contains between 5% and 20% v/v alcohol in water. Using a solution of alcohol in water rather than neat alcohol prevents dehydration of the animal product, which would decrease the efficiency of the collagen composition extraction. The alcohol may be methanol, ethanol, propan-1-ol, propan-2-ol (isopropyl alcohol), butan-1-ol, or butan-2-ol. In one embodiment, the alcohol solution is a solution of isopropyl alcohol.

Where the animal product is washed with both an alkaline solution and a degreasing agent before the animal product is contacted with the acidic solution, the washing with the alkaline solution may occur before or after the washing with the degreasing agent. Typically, the animal product is washed with the alkaline solution prior to washing with the degreasing agent. In one embodiment, the animal product is washed with the alkaline solution prior to washing with an alcohol solution.

The animal product may be washed with water before and/or after each part of the extraction process. For example, the animal product may be washed with water prior to contacting the animal product with the alkaline solution, between contacting the animal product with the alkaline solution and the degreasing agent, and between contacting the animal product with the degreasing agent and the acidic solution.

Further processing steps such as enzymatic digestion or purification of the collagen composition are not required in the extraction method described herein. This reduces the time and resources needed to perform the extraction, compared with methods which require such steps. It also retains the collagen proteins in undigested form and without fragmentation of the protein chains.

(b) Forming

The collagen composition, which may be or comprise an extracted collagen composition, is formed into a collagen gel and the collagen gel is dehydrated to form the biomaterial.

The collagen composition is typically first provided in a suitable solution for forming the biomaterial. For example, the collagen composition may be diluted in water or a buffer solution, or lyophilized collagen composition may be dissolved in water or a buffer solution. A suitable concentration is from 1 mg/mL to 200 mg/mL, e.g., from 10 mg/mL to 100 mg/mL of collagen protein (including collagen, acid-soluble collagen, partially hydrolyzed collagen and fully hydrolyzed collagen).

Advantageously, a collagen composition as used in the present invention, which may comprise an extracted collagen composition, is typically highly soluble in an aqueous solution at a pH of from 5 to 8, for example from pH 6 to 8, from pH 6 to 7 or about pH 7. This means that small volumes of aqueous solvent (such as water) can be used, without requiring the addition of significant volumes of acid to reduce the pH and dissolve the collagen composition. In one embodiment, the collagen composition has a solubility of at least 20 mg, at least 30 mg, at least 40 mg, at least 50 mg of collagen composition per mL of aqueous solvent at a pH of from 5 to 8 and at a temperature of 25° C. Preferably, the collagen composition has a solubility of at least 20 mg, at least 30 mg, or at least 40 mg, or at least 50 mg of collagen composition per mL of aqueous solvent at a pH of from 6 to 7 and at a temperature of 25° C. For collagen compositions which contain collagen, addition of an acidic solvent may be required to achieve dissolution. Any appropriate weak acid or diluted strong acid may be used, for example an aqueous solution of acetic acid, formic acid, or hydrochloric acid. Typically, the collagen is dissolved in an acidic solvent separately to the partially hydrolyzed and/or fully hydrolyzed collagen, and then the collagen solution is added to the solution of partially hydrolyzed and/or fully hydrolyzed collagen to give a combined solution of the collagen composition at a pH of from 5 to 8, preferably at a pH of from 6 to 8 or from 6 to 7. The collagen may be dissolved in an aqueous solution with a pH of less than 5, less than 4, less than 3 or less than 2, preferably less than 3. In one embodiment, the collagen is dissolved in an aqueous solution with a pH of about 2. However, in collagen compositions which contain low amounts of collagen, only a small amount of acidic solution is required. Furthermore, the overall collagen composition is still typically soluble in a solution with a pH of between 5 and 8. Physical agitation such as stirring, mixing and sonication may also be used to aid dissolution. The use of highly soluble collagen compositions in this step is beneficial because smaller volumes of solvent are required, and it is easier to remove the solvent during dehydration.

This is in contrast to collagen mixtures that are typically used in known processes to make biomaterials and which contain only, or mostly, collagen. Such collagen mixtures are typically insoluble at a pH of between 5 and 8, and require much lower pH to dissolve, for example a pH of less than 5. The use of collagen compositions which do not require highly acidic conditions to dissolve means that the solution is easy to neutralize later in the gel forming process. This improves manufacturing efficiency and further reduces the overall amount of solvent required.

As part of the forming step, the collagen composition, which may be or comprise an extracted collagen composition, may be crosslinked using any appropriate protein crosslinking method known in the art. Typically, the collagen composition is contacted with one or more crosslinking agents to form a crosslinkable collagen mixture. The crosslinking agent(s) may be any molecules with di-, tri-, or multifunctional reactive groups that can form crosslinks between collagen molecules. Alternatively, the crosslinking agent(s) may be molecules which can be used in a photoinitiated crosslinking process. The crosslinking agent may be an enzyme. Typically, the crosslinking agent(s) are one or more agents selected from alcohols, aldehydes, amines, azides, carboxylic acids, carbodiimides, chromium salts, epoxides, hydrazides, isocyanates, or sulfhydryls. For example, the one or more crosslinking agents may comprise glutaraldehyde and/or transglutaminase. Appropriate amounts of crosslinking agents are known to those in the art. Typically, from 0.1% to 40% w/w, for example from 1% to 10% w/w of crosslinking agent(s) may be used based on the weight of the collagen composition. Where glutaraldehyde is used as the crosslinking agent, the amount of glutaraldehyde in the crosslinkable composition may be, for example, from 0.5% to 10% w/w, e.g., from 1% to 5% w/w. Alternatively, where the crosslinking agent is an enzyme, typically the enzyme is used in an amount of from 0.1 to 40 U per g of collagen composition, for example from 1 U/g to 10 U/g. Where transglutaminase is used as the crosslinking agent, the amount of transglutaminase may be, for example, from 0.5 U/g to 10 U/g, e.g., from 1 U/g to 5 U/g.

The crosslinkable collagen mixture may also contain a dye or pigment. Thus, in one embodiment, a dye or pigment is added to the collagen composition or crosslinkable collagen mixture. For example, the dye or pigment may be a water-based dye or pigment, an alcohol-based dye or pigment, an acid dye, a direct dye, a mordant dye or a base dye. In one embodiment, the dye is a water-based dye or water-based pigment. Using a dye in this step may help to ensure that the resulting biomaterial is evenly dyed across its thickness.

The crosslinkable collagen mixture may also contain a fatliquoring component, for example a fatliquoring emulsion. Thus, in one embodiment, a fatliquoring component is added to the collagen composition or crosslinkable collagen mixture. The fatliquoring emulsion may include salts of fats, for example sulphonate salts, sulphite salts and/or phosphate salts of triglycerides. Using a fatliquoring emulsion in this step may improve the depth, speed and evenness of penetration of the fatliquor through the biomaterial compared to fatliquoring after gel formation, while still providing beneficial softening and water-repellent properties. Fatliquoring is described further in the description of processing steps.

The crosslinkable collagen mixture may comprise one or more further additives, such as one or more plasticizers. Plasticizers help make the resultant biomaterial soft and flexible. Suitable plasticizers will be known to those in the art. In one embodiment, the plasticizer is glycerol and the crosslinkable collagen mixture comprises glycerol. A plasticizer may, for example, be used in an amount of from 5% to 50% w/w based on the weight of the collagen composition. Where glycerol (also known as glycerin) is used as the plasticizer, the amount of glycerol in the crosslinkable composition may be, for example, from 10% to 40% w/w, e.g., from 20% to 40% w/w.

One or more antifoam agents may also be added to the crosslinkable collagen mixture such that the collagen composition (and crosslinkable collagen mixture) further comprises one or more antifoam agents. The antifoam agent removes bubbles or foams that are formed within the composition, which improves the handling of the composition and helps create a more even biomaterial. Typically, physical agitation such as stirring or agitation is used in combination with an antifoam agent to ensure complete elimination of bubbles and foams. In one embodiment, physical agitation may be applied to the collagen composition and/or crosslinkable collagen mixture which comprises an antifoam agent for at least 10 minutes, at least 20 minutes, at least 30 minutes, or at least 1 hour. A crosslinkable collagen mixture that contains partially hydrolyzed collagen is easier to defoam using an antifoam agent than a mixture which contains only collagen. For example, the present inventors have tried to reproduce Example 2 of EP3205668, which describes the formation of a biofabricated leather from bovine collagen. It was found that the crosslinkable collagen mixture was very thick with large amounts of bubbles that could not easily be removed by routine methods (such as sonication), even at pH 2. It is thought that the presence of partially hydrolyzed collagen in the collagen used in the present invention produces a less thick and viscous mix, which aids defoaming using an antifoam agent.

Suitable antifoam agents will be known to those in the art. In one embodiment, the antifoam agent is a food grade antifoam agent. The antifoam agent may be a silicone-based emulsion, a polypropylene glycol composition or an ethylene oxide (EO) and propylene oxide (PO) copolymer. In one embodiment, the antifoam agent is a silicone-based emulsion. An antifoam agent may, for example, be used in an amount of from 0.001% to 5% w/w where the % w/w means the weight of active ingredient of the antifoam per weight of the total solution. Typically, the amount of antifoam agent added to the solution will depend on the amount of foam produced in the forming process, which may be affected by the various molecular weights of collagen extracted from different sources. The antifoam agent is preferably added in an amount sufficient to remove at least 70%, at least 80%, at least 90% or at least 95% of the bubbles and foams. Typically, the antifoam agent is used in an amount of from 0.001% to 5% w/w, from 0.01% to 3% w/w, from 0.1% to 3% w/w, from 0.1% to 2% w/w, from 0.1% to 1% w/w, or from 0.1% to 0.5% w/w. Preferably, the antifoam agent is used in an amount of no more than 1% w/w. For example, the antifoam agent may be used in an amount of from 0.001% w/w to 1% w/w, from 0.01% w/w to 1% w/w, or from 0.1% w/w to 1% w/w. Where a silicone-based emulsion is used as the antifoam agent, the amount of silicone-based emulsion in the crosslinkable composition may be, for example, from 0.001% to 5% w/w, e.g., from 0.1% to 2% w/w.

The antifoam agent may also improve the properties of the biomaterial and any processed biomaterial produced from the biomaterial. In particular, low concentrations (for example from 0.1% to 3% w/w) of antifoam agent have been found to increase the tensile strength of the resultant biomaterial.

Typically, the crosslinkable collagen mixture has a pH of from 6 to 8 prior to crosslinking, preferably about 7. If the crosslinkable collagen mixture has a pH outside of this range, then appropriate amounts of acid or base may be added to achieve the desired pH. As explained above, the collagen composition used in the present invention is advantageously highly soluble in an aqueous solution at a pH of from 5 to 8, preferably from 6 to 8 or from 6 to 7. Therefore, in one embodiment, no neutralization is required in the forming step to give a crosslinkable mixture with a pH of from 6 to 8. In one embodiment, the forming step involves a neutralization step to raise the pH of the crosslinkable mixture to a range from pH 6 to 8, wherein the neutralization step involves raising the pH of the crosslinkable mixture by no more than 3, no more than 2, or no more than 1.

The crosslinkable collagen mixture may be crosslinked to form the collagen gel. Crosslinking may be achieved by resting the crosslinkable collagen mixture for a period of at least 15 minutes. Typically, the crosslinkable collagen mixture is rested for at least 30 minutes, or at least 1 hour, or at least 2 hours. Alternatively, the crosslinkable collagen mixture is rested for at least 12 hours, or at least 24 hours, or at least 36 hours, or at least 48 hours. In one embodiment, the crosslinkable collagen mixture is rested for at least 48 hours. The crosslinkable collagen mixture may be rested at a temperature of about 1° C. to about 30° C., for example about 1° C. to about 20° C. or about 1° C. to about 10° C. In one embodiment, the crosslinkable collagen mixture is rested at a temperature of about 4° C. In one embodiment, the crosslinkable collagen mixture is rested at a temperature of about 20° C.

The collagen gel is dehydrated to form the biomaterial. The collagen gel may be dehydrated using a suitable dehydrating solvent that is miscible with water such as a ketone or an alcohol. Typically, the collagen gel is dehydrated in acetone or ethanol. Dehydration in a dehydrating solvent such as acetone or ethanol ensures that even dehydration occurs throughout the collagen gel, and in particular prevents one side of the collagen gel drying faster than the other. Alternatively, the collagen gel may be dehydrated using a dehydrator at a temperature of about 25° C. to about 45° C. Typically, the collagen gel is dehydrated using a dehydrator at a temperature of about 30° C. to about 40° C., for example about 35° C. The collagen gel may be dehydrated in the dehydrator for at least 4 hours, at least 6 hours, at least 8 hours, at least 10 hours or at least 12 hours. Typically, the collagen gel is dehydrated in the dehydrator for at least 8 hours, for example about 10 hours. Dehydration in a dehydrator reduces the amount of solvent that is used in the manufacturing process, compared to dehydration processes using a solvent. The water content of the dehydrated collagen gel is typically between 10% and 35%.

The shape of the dehydrated collagen gel (i.e., biomaterial) is not limited and may include any two-dimensional or three-dimensional shape. The shape of the biomaterial may be controlled by, for example, crosslinking the crosslinkable collagen mixture in an appropriately shaped mold. Alternatively, the collagen gel may be shaped and/or reshaped before and/or after dehydration using an appropriate shaping technique. Such shaping may involve bending, folding, stretching, rolling, or cutting the collagen gel or dehydrated collagen gel. Typically, the biomaterial is formed in a sheet, and thus the biomaterial is provided in the form of a sheet. The sheet may be any thickness, but typically the sheet is less than 5 cm thick. Typically, the sheet may be less than 3 cm, 2 cm, 1 cm, 0.5 cm, or 0.1 cm thick. The sheet may be of uniform thickness, or the sheet may have different thicknesses. The sheet may be formed by putting the crosslinkable collagen mixture into an appropriate mold, to provide the desired thickness, and crosslinking to allow gel formation.

Collagen gel formed after crosslinking may be frozen temporarily to enable convenient removal from the mold. The gel is then typically thawed before dehydration.

A biomaterial according to the invention typically has a high tensile strength. For example, the biomaterial may have a tensile strength of at least 5 MPa, or at least 10 MPa, at least 15 MPa, at least 20 MPa or at least 25 MPa. Preferably, the biomaterial has a tensile strength of at least 10 MPa, at least 15 MPa, or at least 20 MPa. In one embodiment, the biomaterial has a tensile strength of from about 5 MPa to about 25 MPa. Tensile strength is typically measured according to the standard method ISO 3376 (2020).

Furthermore, a biomaterial according to the invention is typically semisoft and bendable. The biomaterial generally has the uniform collagen structure throughout its thickness. Additionally, the properties of the biomaterial can be easily altered by the amount and type of crosslinking agent(s) and optional other additives that are used.

(c) Processing

The biomaterial may further be processed to form a processed biomaterial. Thus, methods as described herein may further comprise a processing step (c) after the sheet formation step (b), to form a processed biomaterial. The processed biomaterial is typically a leather-like material.

The processing step may comprise any step or combination of steps which yield a leather-like material. As used herein, a leather-like material refers to a material which has physical properties similar to those of natural leather. Typically, a leather-like material is strong and flexible. The leather-like material may exhibit no cracks when the material is double folded. Although typically the processing steps are carried out on the biomaterial formed from a dehydrated collagen gel, at least the processing steps of fatliquoring and/or dyeing may be incorporated into the gel forming process described above. Where fatliquoring and/or dyeing steps are included in the gel formation process, the biomaterial that is formed after the dehydration of the collagen gel may be a leather-like material without the need for further processing.

The leather-like material may have a lastability of greater than 3 mm, or greater than 5 mm, or greater than 7 mm. In one embodiment, the leather-like material has a lastability of from about 5 mm to about 10 mm. Lastability indicates the amount of distension and strength of the leather grain. Lastability is typically measured using a lastometer according to the standard method ISO 3379 (2015) or DIN 53325.

The leather-like material may have a light fastness such that no change of shade or surface degradation is observed after 10 hours, after 20 hours, after 30 hours or after 40 hours. Light fastness is typically measured according to standard method ISO 105-B02 (2014). Light fastness may also be measured using light at a wavelength of 300 nm-400 nm.

The leather-like material may have a resistance to environmental ageing such that no change in shade or surface degradation is observed after 20 hours, after 40 hours, or after 60 hours of being subjected to an accelerated environmental ageing test. In one embodiment, the accelerated environmental ageing test may comprise subjecting the biomaterial to a temperature of 60° C.±2° C. and a humidity of 90±5% RH.

The leather-like material may have a color fastness to water spotting such that no change of shade or surface degradation is observed after 10 hours, after 13 hours, or after 16 hours. Color fastness to water spotting is typically measured according to the standard method ISO 15700 (1998).

The leather-like material may have a “Martindale” abrasion resistance of at least 3,000 cycles, at least 4,000 cycles, or at least 5,000 cycles (all measured under 9 kPa). In one embodiment, the leather-like material has a “Martindale” abrasion resistance of from about 3,000 cycles to about 6,000 cycles under 9 kPa, as measured using a Martindale abrasion machine. “Martindale” abrasion resistance is typically measured according to the standard method ISO 17076-2 (2011).

The leather-like material may have a “Veslic” color rub fastness such that no degradation is observed after 100 cycles wet and 100 cycles dry, or 125 cycles wet and 125 cycles dry, or 150 cycles wet and 150 cycles dry. “Veslic” color rub fastness is typically measured according to the standard method ISO 11640 (2018).

The leather-like material may have a tensile strength of at least 5 MPa, or at least 10 MPa, at least 15 MPa, at least 20 MPa or at least 25 MPa. In one embodiment, the leather-like material has a tensile strength of from about 5 MPa to about 25 MPa. Tensile strength is typically measured according to the standard method ISO 3376 (2020).

The leather-like material may have a tear strength of at least 5 N, or at least 10 N, or at least 15 N. In one embodiment, the leather-like material has a tear strength of from about 10 N to about 20 N. Tear strength is typically measured according to the standard method ISO 3377-1 (2011) or ISO 3377-2 (2016). In one embodiment, the tensile tear strength may be measured using a low inertia autographic tensile testing machine at a traverse rate of 300±10 mm/minute.

The leather-like material may have a flex resistance such that no surface degradation is observed after 9,000 flexion cycles, after 12,000 flexion cycles, or after 15,000 flexion cycles. Flex resistance is typically measured using a Bally flexometer according to the standard method ISO 5402-1 (2017).

The leather-like material may have low levels of chemical impurities. For example, the leather-like material may contain less than the following amounts of one or more of the following impurities: 75 ppm formaldehyde, 1 ppm chlorophenols, 1 ppm total metal content, 3 ppm Cr(VI), 200 ppm dicyclohexyl phthalate (DCHP), 0.1 ppm dimethyl fumarate, 30 ppm azo dye, 1 ppm polycyclic aromatic hydrocarbons, 1 ppm phthalates, 1 ppm Substance of Very High Concern (SVHC) as defined by the European Chemicals Agency, and 1 ppm organotin compounds.

The processing step may include one or more of fatliquoring, dyeing and drying the biomaterial. Typically, the processing step (c) comprises fatliquoring, dyeing and drying. In one embodiment, the biomaterial is fatliquored, then the fatliquored biomaterial is dyed, then the dyed and fatliquored biomaterial is dried. In another embodiment, the biomaterial is fatliquored, then the fatliquored biomaterial is dried. The processing step may also include mechanically working the biomaterial. As used herein, mechanically working the biomaterial may include bending, folding and/or rolling the biomaterial. In one embodiment, the biomaterial is first fatliquored, then the fatliquored biomaterial is dyed, then the dyed and fatliquored biomaterial is dried, and then the dried biomaterial is worked.

Fatliquoring is a process whereby fats, oils, and/or waxes are fixed to the fibers in a material by coating the material with an emulsion of the fat, oil and/or wax in a solvent. Typically, the fat, oil and/or wax is an oil such as vegetable oil, castor oil, pine oil, lanolin, or fish oil. For example, fatliquoring may include contacting the biomaterial or processed biomaterial with an emulsion of vegetable oil in acetone. In the processing of natural leather, fatliquoring is used to regrease the surface of the leather to increase softness and flexibility. Fatliquoring also adds water-repellent properties. Fatliquoring a biomaterial formed from a marine collagen composition may produce a rigid and brittle material. However, this rigid and brittle fatliquored material can still be made into a leather-like material by further processing steps, in particular by further dyeing, drying, treatment with an alcohol solution and/or mechanically working the material.

Any suitable dye or pigment may be used for dyeing. Suitable leather dyes and pigments are known in the field and may include water-based dyes and pigments, alcohol-based dyes and pigments, acid dyes, direct dyes, mordant dyes, or base dyes. In one embodiment, the dye is an alcohol-based dye, in particular an ethanol-based dye. Treatment with an alcohol solution may be used as well as, or instead of, a dying step. Typically, the alcohol solution is an ethanol solution.

The drying step typically comprises drying the biomaterial at a temperature of at least 30° C., or at least 40° C., or at least 50° C. Drying may help to soften the rigid biomaterial that is produced after fatliquoring. The drying step may be performed in a dehydrator. Typically, the water content of a dried biomaterial may be between 5% and 25%.

Typically, processing also includes a finishing and/or coating step to give the biomaterial the desired aesthetics. Appropriate finishing chemicals and formulations are known to those skilled in the art, and may include water repellent chemicals, beeswax, or synthetic polymers. If a water-based finishing formulation is used, the finished biomaterial must be dried according to the description above, in order to remove the aqueous solvent.

Processing may also include any other process which is typically applied to natural leather including rehydrating, splitting, shaving, neutralization, filling, setting, conditioning, softening, or buffing.

### MANUFACTURE OF AN ARTICLE

The biomaterial or processed biomaterial may be used in the manufacture of an article comprising the biomaterial or processed biomaterial. Manufacturing may include any step of shaping and/or cutting the biomaterial or processed biomaterial. The article may be any article which can usefully be made out of a leather-like material, such as accessories, shoes, and furniture.

### EXAMPLES

### Example 1

A leather-like processed biomaterial was made according to the method below.

(a) Extraction

Cod skin (80 g, wet mass) was cut into 3 cm×5 cm strips and cleaned with deionized water. 250 mL of 0.1 M NaOH was added and stirred for 2 hours at room temperature. (The NaOH was changed after 1 hour). The skin was washed with water.

10% v/v isopropyl alcohol in water (200 mL) was added and stirred for 1 hour at room temperature. The skin was washed with water.

200 mL of 1 M AcOH was added and stored at 4° C. overnight. The extract was then separated. Another 200 mL of 1 M AcOH was added to the fish skin and stored at 4° C. overnight again.

The two extracts were combined and lyophilized to afford a white amorphous solid. Yield: 100 g/kg (dry mass fish skin).

(b) Forming

100 mg of fish skin extract from step (a) was dissolved in 2 mL water at room temperature. 30% w/w glycerol and 2% w/w glutaraldehyde were added and mixed well. The solution was transferred into a mold and rested at 4° C. for two days for gel formation.

The gel was put into the freezer for 1 hour and removed from the mold. After thawing, the gel was put into 50 mL of acetone for dehydration (shaker table 40 RPM for 48 hours, fresh acetone was exchanged after 24 hours).

After dehydration, the material was semisoft and bendable.

(c) Processing

The material was subjected to fatliquoring: the sample was immersed in a solution of 20% v/v vegetable oil in acetone and put on a shaker table at 40 RPM for 8 hours. The sample was removed from the solution and the excess amount of oil solution was wiped away. After fatliquoring, the material became very rigid.

The fatliquored material was then dyed, by immersing in an ethanol-based black leather dye for 1 hour. The excess amount of dye was wiped away after dyeing.

The dyed material was then dried in a dehydrator at 40° C. for 5 hours.

The sample became leather-like after bending multiple times.

### Example 2

A biomaterial was made according to the method of Example 1 except that the extraction step (a) was omitted and 100% fish gelatin was used as the starting material for step (b) instead of the fish skin extract.

After completion of the processing step (c), the resulting material was softer and more elastic than the material produced in Example 1.

### Example 3

A biomaterial was made according to the method of Example 1 except that the dyeing process in the processing step (c) was omitted. It was observed that the rigid biomaterial that was produced after fatliquoring became less rigid after drying.

### Example 4

A biomaterial was made according to the method of Example 1 except that the dyeing process in the processing step (c) was combined with forming step (b). In this example, a water-based dye is mixed with collagen solution prior to gel formation. It was observed that the resulting biomaterial is evenly dyed across the thickness of the biomaterial.

### Example 5

The following example describes a method for making a biomaterial using partially hydrolyzed collagen, and optionally fully hydrolyzed collagen, but with no collagen.

Partially hydrolyzed collagen and fully hydrolyzed collagen were purchased from Louis Francois and InnerVita.

Gel Formation

The protein mixture (2.5 g) was dissolved in 50 mL degassed Type II water at the temperature around 50° C.-60° C. After fully dissolved, the protein mixture was sonicated for 30 seconds and cooled down to a room temperature (20° C.-25° C.). 0.75 g glycerin (Intralabs) was added into the protein mixture and stirred for 1-2 minutes until fully dissolved. 0.2 mL glutaraldehyde (Alfa Aesar, 2% w/w of protein) was added into the solution at the room temperature and the solution was mixed for 1-2 min before pouring into a 12 cm×12 cm or 25 cm×25 cm mold. Hydrogel formed at room temperature after 30 min.

Dehydration

The collagen hydrogel was dehydrated in a dehydrator at 35° C. for 10 hours and was peeled off from the mold.

### Example 6

The following example describes a method for making a biomaterial using partially hydrolyzed collagen, and optionally fully hydrolyzed collagen, with collagen.

Freeze dried Type I collagen sheet isolated from porcine skin and bovine tendon was purchased from Wuxi BIOT Bio-technology Co. Ltd. Marine collagen was extracted from cod skin following established procedures. Partially hydrolyzed collagen and fully hydrolyzed collagen were purchased from Louis Francois and InnerVita. XIAMETER AFE-1530 antifoam agent (silicone-based emulsion) was purchased from the Dow Chemical Company.

Gel Formation

0.25-0.5 g collagen sheet was cut into small pieces and weighed in preparation for use. The partially hydrolyzed collagen and/or partially hydrolyzed collagen/fully hydrolyzed collagen mixture (2-2.25 g) was dissolved in 25 mL type II degassed water at 50° C.-60° C. The protein solution was cooled down to a room temperature (20° C.-25° C.). The prepared collagen pieces were added in a 25 mL 0.01 M HCl aqueous solution. After collagen was adequately dissolved, 0.75 g of glycerin was added into the solution. In order to defoam the solution, 0.2%-0.4% w/w antifoam emulsion was added and the solution was further stirred for at least 30 min until the foams were fully eliminated. The protein solutions were combined and then neutralized by adding aliquots of 5M NaOH aqueous solution. 0.2 mL glutaraldehyde (2% w/w) was added dropwise, and the solution was stirred for 1-2 min before pouring into a 12 cm×12 cm or 25 cm×25 cm mold. Hydrogel formed at room temperature after 30 min.

Dehydration

The collagen hydrogel was dehydrated in a dehydrator at 35° C. for 10 hours and was peeled off from the mold.

### Example 7

Biomaterials using various collagen-containing components were prepared according to the methods of Examples 5 and 6. The method of Example 5 was used where the composition contained no collagen, and the method of Example 6 where the composition contained collagen. The tensile strength of the biomaterials was tested using a tensile strength tester Instron Model 34SC-05, following the standard method ISO 3376:2020.

The composition and tensile strength of the biomaterials is described in Table 1.

TABLE 1
COMPOSITION OF BIOMATERIALS

| | | Partially | Fully | | Glutaralde | | Tensile |
|---|---|---|---|---|---|---|---|
| | Collagen | Hydrolyzed | Hydrolyzed | Glycerin | Hyde | Antifoam | Strength |
| Entry | % | Collagen % | Collagen % | w/w % | w/w % | w/w % | MPa* |
| 1 | 100% | — | — | 30 | 2 | — | E |
| | Marine | | | | | | |
| 2 | 100% | — | — | 30 | 2 | 0.12 | E |
| | Bovine | | | | | | |
| 3 | 50% | 50% | — | 30 | 2 | 0.12 | D |
| | Bovine | Marine | | | | | |
| 4 | 50% | 50% | — | 30 | 2 | 0.03 | A |
| | Porcine | Marine | | | | | |
| 5 | 20% | 80% | — | 30 | 2 | 0.12 | B |
| | Porcine | Marine | | | | | |
| 6 | 10% | 90% | — | 30 | 2 | 0.12 | D |
| | Bovine | Marine | | | | | |
| 7 | 10% | 90% | — | 30 | 2 | 0.12 | B |
| | Porcine | Marine | | | | | |
| 8 | 10% | 90% | — | 30 | 2 | 0.6 | B |
| | Porcine | Marine | | | | | |
| 9 | — | 100% | — | 30 | 2 | — | A |
| | | Marine | | | | | |
| 10 | — | 100% | — | 30 | 2 | — | A |
| | | Porcine | | | | | |
| 11 | — | 100% | — | 30 | 2 | 0.3 | A |
| | | Marine | | | | | |
| 12 | — | 50% | — | 30 | 2 | 0.001 | A |
| | | Marine | | | | | |
| | | 50% | | | | | |
| | | Porcine | | | | | |
| 13 | — | 90% | 10% | 30 | 2 | — | A |
| | | Marine | Marine | | | | |
| 14 | — | 80% | 20% | 30 | 2 | — | B |
| | | Marine | Marine | | | | |
| 15 | 10% | 80% | 10% | 30 | 2 | 0.12 | D |
| | Bovine | Marine | Marine | | | | |
| 16 | 10% | 80% | 10% | 30 | 2 | 0.12 | A |
| | Porcine | Marine | Marine | | | | |

*A: >20 MPa; B: 16-20 MPa; C: 11-16 MPa; D: 6-11 MPa; E: <6 MPa

Conclusion

The results in Table 1 demonstrate that biomaterials formed from collagen compositions containing partially hydrolyzed collagen (entries 3 to 16) show improved tensile strength compared to biomaterials formed from collagen compositions containing collagen as the only collagen-containing component (entries 1 and 2).

The results also show that the addition of antifoam agent at a low concentration has successfully prevented foam formation during the gel forming process, thus increasing the mechanical strength of the biomaterials.

Marine collagen is a particularly advantageous collagen source. For example, a biomaterial made from 100% marine collagen (entry 1; 5.4 MPa) showed improved tensile strength over a biomaterial made from 100% bovine collagen (entry 2; 4.1 MPa).

### Example 8

Biomaterials using various collagen-containing components were prepared according to the methods of Examples 5 and 6. A fatliquor emulsion (40% w/w of protein) was added to the collagen composition during gel formation. The results are shown in Table 2.

TABLE 2
TENSILE STRENGTH OF BIOMATERIALS
MADE FROM COLLAGEN COMPOSITION
COMPRISING A FATLIQUOR EMULSION

| | | Tensile |
|---|---|---|
| Entry | Protein | strength, MPa |
| 1 | 100% partially hydrolyzed marine collagen | A |
| | with 40% fatliquor emulsion (Trupon LP2 | |
| | from Trumpler) | |
| 2 | 100% partially hydrolyzed marine collagen | A |
| | with 40% fatliquor emulsion (Trupon LP2 | |
| | from Trumpler) 20% glycerin | |

### Example 9

Biomaterials using various collagen-containing components were prepared according to the methods of Examples 5 and 6, except that transglutaminase (protein-glutamine γ-glutamyltransferase, EC 2.3.2.13, provided by Stabizym TGL-100) was used as the crosslinking agent instead of glutaraldehyde.

TABLE 3
COMPOSITION OF BIOMATERIALS CONTAINING
TRANSGLUTAMINASE AS CROSSLINKING AGENT

| | | Partially | Fully | | Transglu- | | Tensile |
|---|---|---|---|---|---|---|---|
| | Collagen | Hydrolyzed | Hydrolyzed | Glycerin | taminase | Antifoam | Strength |
| Entry | % | Collagen % | Collagen % | w/w % | U/g | w/w % | MPa |
| 1 | — | 100% | — | 30 | 2 | — | A |
| | | Marine | | | | | |
| 2 | — | 100% | — | 30 | 2 | 0.001 | A |
| | | Marine | | | | | |
| 3 | — | 100% | — | 30 | 4 | — | A |
| | | Marine | | | | | |
| 4 | — | 100% | — | 30 | 4 | 0.001 | A |
| | | Marine | | | | | |
| 5 | — | 90% | 10% | 30 | 4 | 0.001 | B |
| | | Marine | Marine | | | | |
| 6 | — | 80% | 20% | 30 | 4 | 0.001 | C |
| | | Marine | Marine | | | | |

Discussion

The addition of antifoam increased the tensile strength of the biomaterial. For example, the biomaterial made from the composition at entry 2 (0.001% w/w antifoam; 31.5 MPa) showed improved tensile strength over entry 1 (no antifoam; 25.6 MPa).

### Example 10

Biomaterials using various collagen-containing components were prepared according to the method of Example 9. A water-based dye (Metropolitan Leather) was added to the solution during gel formation. The results are shown in Table 4.

TABLE 4
TENSILE STRENGTH OF BIOMATERIALS
MADE FROM COLLAGEN COMPOSITION
COMPRISING A WATER-BASED DYE

| | | Tensile |
|---|---|---|
| Entry | Protein | strength, MPa |
| 1 | 100% partially hydrolyzed marine collagen, | A |
| | 2 U/g transglutaminase, 0.001% antifoam, | |
| | 0.1 mL/L water-based dye | |

1. A biomaterial comprising a dehydrated collagen gel, wherein the collagen gel comprises a collagen composition comprising (i) partially hydrolysed collagen, and (ii) collagen and/or fully hydrolysed collagen, wherein
the collagen composition comprises at least 30% by weight of partially hydrolysed collagen.

2. The biomaterial according to claim 1, wherein the collagen composition comprises at least 5% by weight of one or a mixture of collagen and/or fully hydrolysed collagen.

3. The biomaterial according to claim 1 or claim 2, wherein the collagen composition comprises from 50% to 95% by weight of partially hydrolysed collagen.

4. The biomaterial according to any one of the preceding claims, wherein the collagen composition comprises from 70% to 95% by weight of partially hydrolysed collagen.

5. The biomaterial according to claim 4, wherein the collagen composition comprises from 80% to 95% by weight of partially hydrolysed collagen.

6. The biomaterial according to claim 4, wherein the collagen composition comprises from 5% to 30% by weight of one or a mixture of collagen and/or fully hydrolysed collagen.

7. The biomaterial according to claim 5, wherein the collagen composition comprises from 5% to 20% by weight of one or a mixture of collagen and/or fully hydrolysed collagen.

8. The biomaterial according to any one claims 1 to 4, wherein the collagen composition comprises from 5% to 30% by weight of fully hydrolysed collagen, preferably wherein the collagen composition comprises from 5% to 20% by weight of fully hydrolysed collagen.

9. The biomaterial according to any one of the preceding claims, wherein at least one of the partially hydrolysed collagen, collagen and fully hydrolysed collagen is extracted from a marine product.

10. The biomaterial according to claim 9, wherein the marine product is a fresh-water fish product, a salt-water fish product, an invertebrate product or an arthropod product.

11. The biomaterial according to claim 9 or claim 10, wherein the partially hydrolysed collagen is partially hydrolysed marine collagen.

12. The biomaterial according to any one of the preceding claims, wherein the collagen gel comprises an extracted collagen composition, and wherein the extracted collagen composition comprises collagen.

13. The biomaterial according to claim 12, wherein the extracted collagen composition is extracted from a marine product.

14. The biomaterial according to any one of the preceding claims, wherein the collagen composition further comprises an antifoam agent.

15. The biomaterial according to claim 14, wherein the antifoam agent is present in an amount from 0.001% to 5% w/w.

16. The biomaterial according to claim 15, wherein the antifoam agent is present in an amount from 0.1% to 2% w/w.

17. A method for producing the biomaterial according to any one of claims 1 to 16, the method comprising:
a) forming the collagen composition into a collagen gel; and
b) dehydrating the collagen gel to form the biomaterial.

18. The method according to claim 17, wherein the forming step comprises adding a fat-liquoring component and/or a dye or pigment to the collagen composition.

19. The method according to claim 17 or claim 18, wherein the method further comprises processing the biomaterial to form a processed biomaterial.

20. A leather-like processed biomaterial comprising a biomaterial according to any one of claims 1 to 16.
